(19) United States
(12) Patent Application Publication
(10) Pub. No.: US 2015/0190093 A1
(43) Pub. Date: Jul. 9, 2015

(54) PORTABLE ELECTRONIC DEVICE

(71) Applicant: EMINENT Electronic Technology Corp. Ltd., Hsinchu (TW)

(72) Inventors: Tom Chang, Taipei City (TW); Kao-Pin Wu, New Taipei City (TW); Chih-Jen Fang, Tainan City (TW); Yuan-Shun Yeh, Zhubei City (TW); De-Cheng Pan, Lukang Township (TW)

(73) Assignee: EMINENT Electronic Technology Corp. Ltd.

(21) Appl. No.: 14/573,589

(22) Filed: Dec. 17, 2014

Related U.S. Application Data
(60) Provisional application No. 61/923,239, filed on Jan. 3, 2014.

(30) Foreign Application Priority Data
Oct. 31, 2014 (TW) 103137741

(51) Int. Cl. A61B 5/00; G09G 3/34; A61B 5/024; A61B 5/02; A61B 5/1455; A61B 5/021

(52) U.S. Cl. CPC A61B 5/6898; A61B 5/14551; A61B 5/021; A61B 5/02416; A61B 5/02007; A61B 5/4845; A61B 5/7278; G09G 3/3413; G09G 2360/144; G09G 2320/066; G09G 2320/0666

(57) ABSTRACT

A portable electronic device includes a switching unit, a light source, a light detector and control unit. The user utilizes the switching unit to select the detection mode. The light is emitted from the light source to the object to generate a reflected light. The light detector detects the reflected light to generate a light detecting signal for the control unit to calculate a corresponding result based on the selected mode. Therefore, single portable electronic device can achieve multiple functions and is more convenient for utilization.

### CROSS-REFERENCE TO RELATED APPLICATIONS

This application claims the benefit of United States provisional application filed on Jan. 3, 2014 and having application Ser. No. 61/923,239, the entire contents of which are hereby incorporated herein by reference

This application is also based upon and claims priority under 35 U.S.C. 119 from Taiwan Patent Application No. 103137741 filed on Oct. 31, 2014, which is hereby specifically incorporated herein by this reference thereto.

### BACKGROUND OF THE INVENTION

1. Field of the Invention

The present invention relates to a portable electronic device, especially to a portable electronic device that uses light sources with light detectors to execute multi-functions.

2. Description of the Prior Arts

With the progress of the technology, portable electronic devices are easily carried so that many different portable electronic devices with different functions are developed to allow the user to utilize those functions anytime anywhere. Besides the portable phones with communicating functions, other portable electronic devices such as blood sugar detecting devices, heart rate detecting devices, blood oxygen saturation detecting devices, breath ethanol concentration analyzing devices, remote controller and so on are also developed. Those portable electronic devices use light sources and light detectors to achieve their own functions. However, the user needs to have multiple different portable electronic devices for different functions so that the user has to cost much to buy those different portable electronic devices with different functions.

To overcome the shortcomings, the present invention provides a portable electronic device to mitigate or obviate the aforementioned problems.

### SUMMARY OF THE INVENTION

The main objective of the present invention is to integrate the functions so that the portable electronic device executes different functions with the same elements.

To achieve the aforementioned objective, the present invention provides a portable electronic device comprising:

a first switching unit to switch the portable electronic device to a blood detection mode;

a first light source to provide a first beam, wherein under the blood detection mode, the first beam is emitted to a first object to be reflected as a reflected light, and the first object is a part of an user's body;

a light detector having at least one light detecting unit, wherein under the blood detection mode, the light detector detects the reflected light to generate a blood detecting signal; and

a control unit connecting to the light detector, wherein under the blood detection mode, the control unit calculates the user's blood pressure based on the blood detecting signal.

Further, the portable electronic device further comprises a second switching unit to switch the portable electronic device to an ultraviolet detection mode. Under the ultraviolet detection mode, the light detector detects an ultraviolet in an ambient light around a periphery environment of the portable electronic device to generate an ultraviolet detecting signal, and the control unit calculates the intensity of the ultraviolet in the ambient light based on the ultraviolet detecting signal.

Moreover, the portable electronic device further comprises a display having a backlight module and a second switching unit to switch the portable electronic device to a color detection mode. The light detector has three light detecting units to respectively detect red light, green light and blue light. Under the color detection mode, the light detecting units respectively detect red light, green light and blue light in an ambient light around a periphery environment of the portable electronic device to generate a color detecting signal, and the control unit adjusts the illumination color contrast of the backlight module of the display based on the color detecting signal.

The present invention has following advantages. Using the same element to achieve different functions allows the portable electronic device to provide different functions as required separately or simultaneously. Then the user only needs to buy a single portable electronic device and can also have various functions. Therefore, the portable electronic device not only can save cost but is more convenient.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a portable electronic device in accordance with the present invention;

FIG. 2 is a block diagram of the circuit of the light detector of the portable electronic device in FIG. 1;

FIG. 3 is a flow chart of a first embodiment of a performing method of the portable electronic device in FIG. 1;

FIGS. 4A and 4B are illustrating operational views of the light source and light detector of the portable electronic device in FIG. 1, shown that the user uses finger to execute the light source and light detector;

FIG. 5 is a flow chart of a second embodiment of a performing method of the portable electronic device in FIG. 1; and

FIG. 6 is a flow chart of a third embodiment of a performing method of the portable electronic device in FIG. 1.

### DETAILED DESCRIPTION OF THE PREFERRED EMBODIMENTS

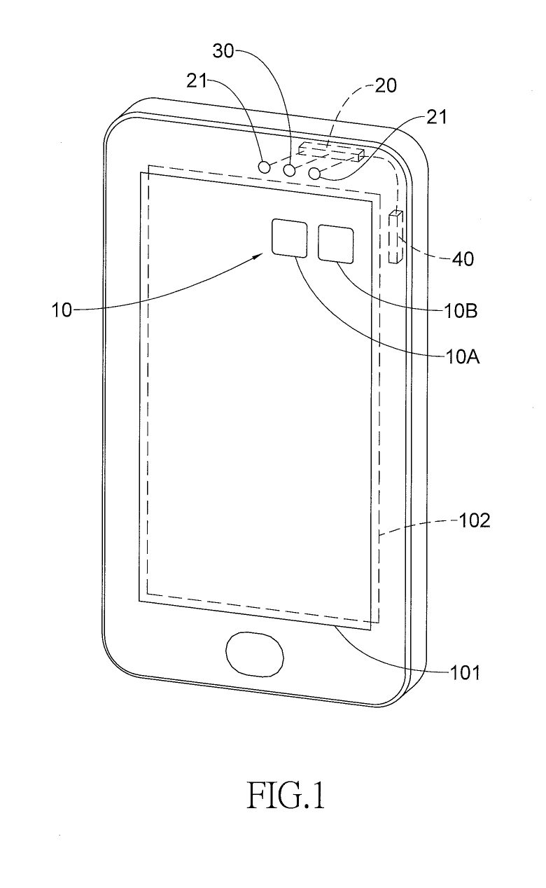

With reference to FIG. 1, a portable electronic device in accordance with the present invention has at least one switching unit 10, a light detector 20, at least one light source 30 and a control unit. The portable electronic device in accordance with the present invention may be any kind of electronic devices such as mobile phones, tablet PC, smart watches, smart glasses and so on, wherein a mobile phone is illustrated as an example in the following drawings.

The switching unit 10 may be an icon shown on a display 101. The display 101 may have a backlight module 102. Different switching units 10 such as a first switching unit 10A and a second switching unit 10B correspond to different function modes. When each switching unit 10 is actuated such as the user's finger touches the icon, the portable electronic device as described enters corresponding function mode. Besides displayed as an icon, the switching unit 10 may also be a real button mounted on the portable electronic device as described to be pressed by user to switch different function modes.

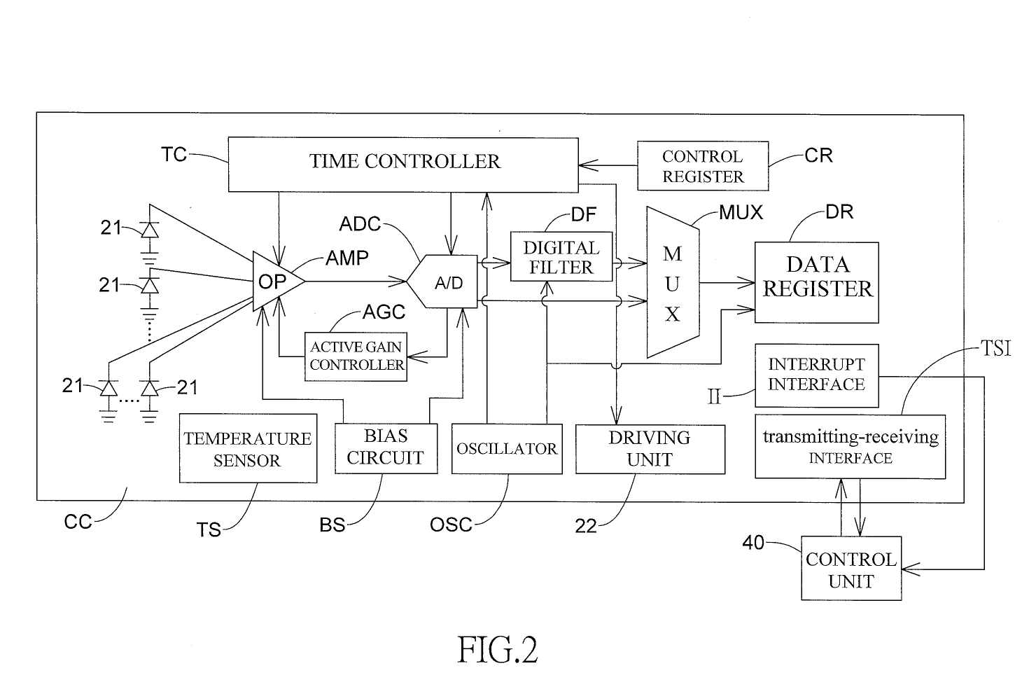

With reference to FIGS. 1 and 2, the light detector 20 has at least one light detecting unit 21 and at least one driving unit 22. Each driving unit 22 is connected to and drives a corresponding light source 30. The light source 30 emits light through the holes on the portable electronic device as described. The light detecting unit 21 receives the lights through the holes on the portable electronic device as described to generate light detecting signal. The amount and the relating locations of the light detecting units 21 and the light sources 30 are regarded as the functions provided by the portable electronic device as described. Further, the light detecting units 21 and the light sources 30 may be integrated in a semiconductor package. FIG. 1 shows a light source 30 with two light detecting units 21 and the light source 30 is mounted between the light detecting units 21. However, FIG. 1 only shows one embodiment of the present invention and the present invention is not limited thereto. In a preferred embodiment, the light detector 20 comprises multiple light detecting units 21, a time controller TC, an amplifier AMP, an active gain controller AGC, an analog-to-digital converter ADC, a digital filter DF, a multiplexer MUX, a control register CR, a data register DR, an interrupt interface II, a transmitting-receiving interface TSI, a light source controller LC, an oscillator OSC, a bias circuit BC and a temperature sensor TS. The light detecting unit 21 detects light to generate said light detecting signal. The amplifier AMP amplifies the light detecting signal and the amplification of the amplifier AMP is adjusted by the active gain controller AGC. The active gain controller AGC adjusts the amplification of the amplifier AMP and the integration time of the light detecting unit 21 for the light detecting signal to reach the desired brightness. The analog-to-digital converter ADC converts the amplified light detecting signal to a digital signal. The digital filter DF filters noises. The time controller TC controls the time sequences of the elements in the light detector 20. The temperature sensor TS detects the temperature. The bias circuit BS is a bias voltage source of the analog circuit. The oscillator OSC provides the clock signal. The light source controller LC controls the said light sources 30. The control register CR and the data register DR respectively storage commands and detecting results. The transmitting-receiving interface TSI transmits and receives the commands and the data. The interrupt interface II notifies the control unit 40 about the condition of the storage space to determine the data whether transmitted or received.

The portable electronic device as described has different performing processes based on the selected modes of the user.

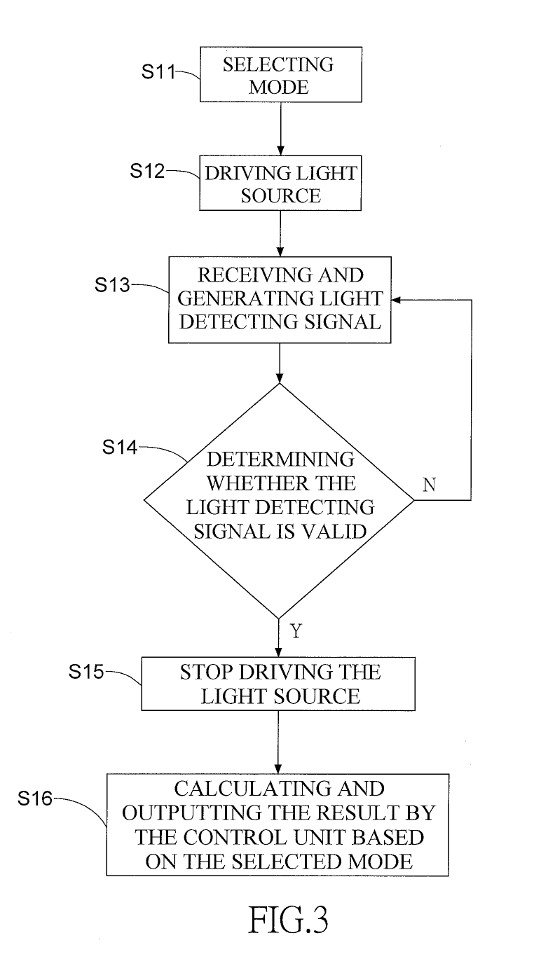

With reference to FIGS. 1 to 3, one of the performing processes comprises following steps. The user selects desired mode through the switching unit 10 (S11). The control unit 40 sends commands to the light detector 20 to ask the corresponding driving unit 22 to drive the corresponding light source 30 (S12). The light of the light source 30 is emitted to the object to generate a reflected light. The reflected light is received by the corresponding light detecting unit 21 to generate light detecting signal (S13). The light detecting signal is stored in the data register DR. When the controller 40 reads the light detecting signal stored in the data register DR, the controller 40 determines whether the light detecting signal is valid (S14). If the light detecting signal is not valid, then return to step S13. If the light detecting signal is valid, the light source 30 is stopped being driven (S15). The control unit 40 calculates the parameter based on the received light detecting signal (S16). Then the calculating result is output based on the selected mode.

In specific, the blood are sent to the whole body when the heart is systole and the blood flows back to the heart when the heart is diastole. Therefore, observing the blood flow can have the related biological parameter of the heart. When a blood pressure detection mode, a heart rate detection mode, a sclerosis vascularis detection mode or a vessel occlusion detection mode is selected, the light detecting unit 21 of the light detector 20 receives a reflected light of the first light beam to generate a blood detecting signal. When the blood pressure mode is proceeded, the control unit 40 calculates the blood pressure through a corresponding algorithm based on the blood detecting signal. When the heart rate mode is proceeded, the control unit 40 uses Fourier transformation to have a spectrum based on the blood detecting signal. Then the spectrum is used to calculate the heart rate. When the sclerosis vascularis detection mode or vessel occlusion detection mode is selected, the control unit 40 uses the know way to process the blood detecting signal to have the arterial stiffness index and the vessel obstruction index. Therefore, the light detecting signal generated by the light detector 20 is calculated through different algorithms to have different biological indexes. The processes can be achieved through different ways and are known in the art so that the details are not described here. In a preferred embodiment, the light source 30 may emits beam having wavelength between 400 to 1000 nm, especially 550 nm. The first switching unit 10A can simultaneously actuate blood detection mode, the heart rate detection mode, the sclerosis vascularis detection mode and the vessel occlusion detection mode can be executed. In other preferred embodiment, different switching units 10 can be used to actuate the aforementioned detection mode.

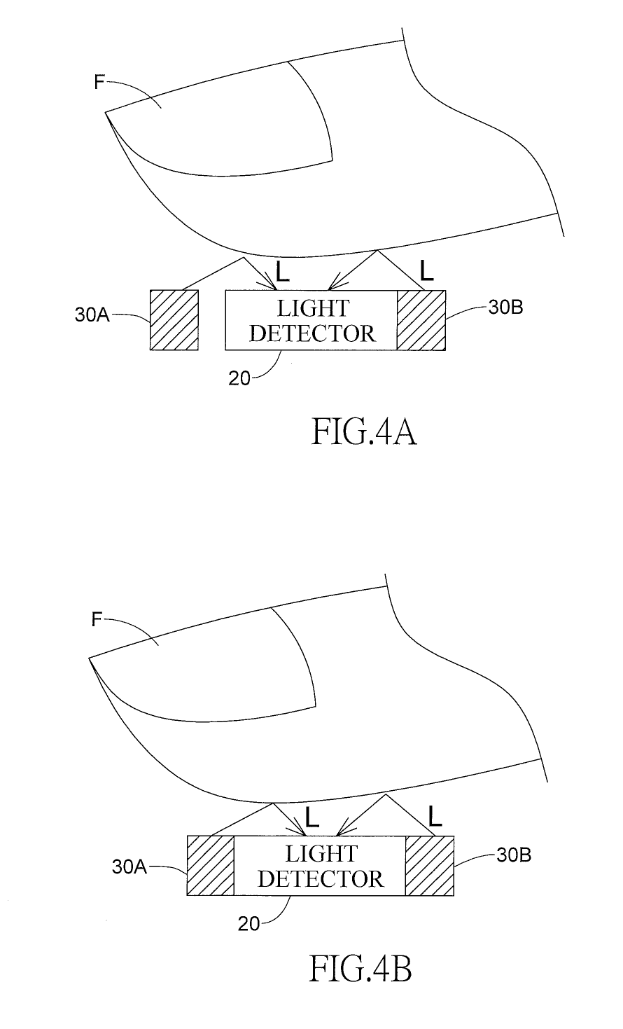

Further, the portable electronic device as described may provide the blood oxygen saturation detection mode and may comprise a first light source 30A and a second light source 30B as shown in FIGS. 4A and 4B to emit different beams with different wavelengths, preferably be a light source emitting red light with 660 nm wavelength and a light source emitting infrared ray with 940 nm. With the second switching unit 10B to select the blood oxygen saturation detection mode, the first light source 30A and the second light source 30B are driven. The first and second beams L from the first and second light sources 30A, 30B are emitted to the user's finger F. the light detector 20 receives the reflected light of the first and second beams to generate a blood oxygen saturation signal. Since the energies of different beams with different wavelengths emitted to bloods with different blood oxygen saturations are different, the control unit 40 uses the known way to process the blood oxygen saturation signal and then the blood oxygen saturation index is obtained. In a preferred embodiment, the light detector 20 has different light detecting units 21 to respectively receive the first and second beams with different wavelengths simultaneously. In another preferred embodiment, the light detector 20 has only on light detecting unit 21 and the first and second light sources 30A, 30B are switched on alternatively. Then the light detecting unit 21 receives the first and second beams with different wavelengths in sequence. The first light source 30A may be independent to the light detector 20 while the light detector 20 may be integrated with the second light source 30B in a semiconductor package as shown in FIG. 4A. The first and the second light sources 30A, 30B and the light detector 20 may be integrated in a semiconductor package as shown in FIG. 4B.

Moreover, when an breath ethanol concentration analyzing mode is selected via the second switching unit 10B, the light source 30 emits a first beam. The first beam is emitted to the user's breath to have a reflected light. The light detecting unit 21 of the light detector 20 receives the reflected light to generate an ethanol concentration signal. The control unit 40 analyzes the ethanol concentration signal to compare with the pre-stored data to calculate the ethanol concentration. The pre-stored data may be light detecting signal generated by the normal breath without ethanol. In a preferred embodiment, the light source 30 may emit infrared ray with 850 nm. Since the ethanol absorbs the infrared ray, the infrared ray is absorbed when the breath passed by the infrared ray emitted by the light source 30 contains ethanol. Then the value output by the light detector 20 is lower than the value that resulted from the normal breath without ethanol. Therefore, using said analyzing way, the ethanol concentration is calculated.

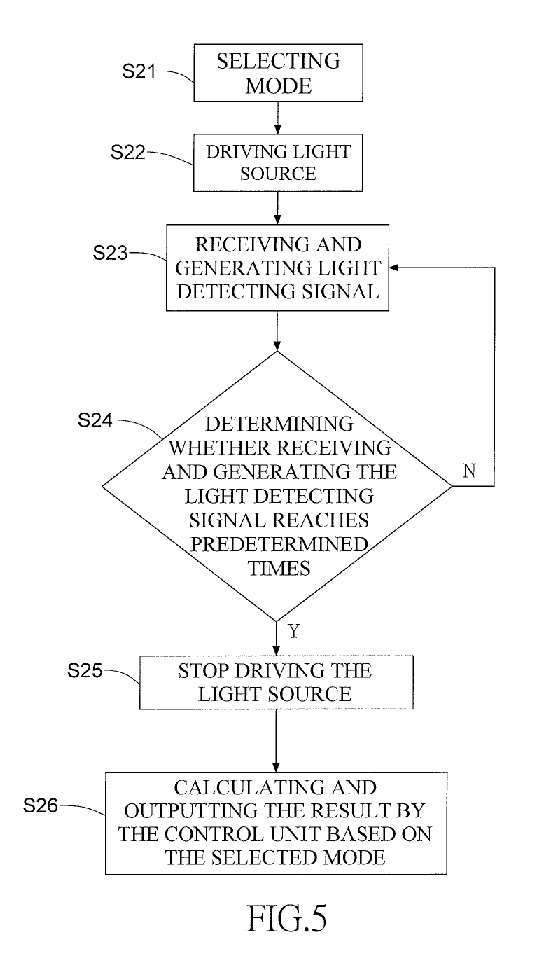

With reference to FIGS. 1, 2 and 5, another performing process comprises following steps. The user selects desired mode through the switching unit 10 (S21). The control unit 40 sends commands to the light detector 20 to ask the corresponding driving unit 22 to drive the corresponding light source 30 (S22). The light of the light source 30 is emitted to the object to generate a reflected light. The reflected light is received by the corresponding light detecting unit 21 to generate light detecting signal (S23). The light detecting signal is stored in the data register DR. When the controller 40 reads the light detecting signal stored in the data register DR, the controller 40 determines whether receiving the light detecting signal is reached predetermined time (S24). If the predetermined time is not reached yet, then return to step S23. If the predetermined time is reached, the light source 30 is stopped being driven (S25). The control unit 40 calculates the parameter based on the received light detecting signals (S26). Then the calculating result is output based on the selected mode.

In specific, the second switching unit 10B is actuated to select a proximity sensing mode, or the portable electronic device as described directly executes the proximity sensing mode while specific application programs are executed such as voice communication. The proximity sensing mode is used to detect whether any objects are approaching the surface of the portable electronic device. The light source 30 provides a first beam. When objects is approaching, the first beam emitted to the object to have a reflected light. The light detecting unit 21 of the light detector 20 receives the reflected light of the first beam to generate a proximity sensing signal. When the object is closer, the intensity of the reflected light is larger so that the control unit 40 can determine whether any objects are approaching based on the proximity sensing signal. In a preferred embodiment, the portable electronic device automatically enters the proximity sensing mode during voice communication. Then when large object such as human face approaches, the touch detection is closed due to the execution of the proximity sensing mode.

Further, the portable electronic device may provide gesture detection mode with one light source 30 and one light detecting unit 21. Through the second switching unit 10B to select the gesture detection mode, the light source 30 emits beam. whether the light detecting unit 21 receives the reflected light determines whether gesture is occurred. The gesture mode may be used as a switch to switch on or off specific function of the portable electronic device.

Moreover, the portable electronic device as described may provide the gesture detection mode and may comprise a first light source 30A and a second light source 30B as shown in FIG. 4A with a light detecting unit 21, or may comprise a light source 30 with two light detecting units 21. If the portable electronic device comprises two light sources and one light detecting unit, the first and second light sources 30A, 30B emits beam to the finger F to generate reflected lights when the finger F moves. Since the light sources 30A, 30B emit in turns, the light detecting unit 21 of the light detector 20 receives the reflected lights from the finger F corresponding to the light sources 30A, 30B based on the emitting order of the light sources 30A, 30B. Based on the emitting order of the light sources 30A, 30B and the detecting result of the light detecting unit 21, two waves of the reflected light with different phases are obtained. For example, the light detector 20 receives the signal having a first wave when the first light source 30A emits. The light detector 20 receives the signal having a second wave when the second light source 30B emits. The control unit 40 determines the movement of the finger F based on the phase relation of the two reflected lights such as ahead or behind. If the portable electronic device comprises one light source and two light detecting units, the light source 30 emits beam to the finger F to generate a reflected light when the finger F moves. When the finger F moves closely to one of the light detecting units, this light detecting units receives the beam of the reflected light with stronger intensity so that the information of the reflected lights received by each light detecting unit 21 can be used to determine the position and the movement of the finger F. The reflected light received by each light detecting unit 21 is converted to the output of the light detector 20 for the control unit 40 to determine the movement of the finger F. The gesture may be operated by fingers, palms, or hands. For example, a light detecting unit matrix of the light detector 20 may be used to detect image and the position and the moving trace of the finger can be calculated. Using the first and second light sources 30A, 30B with one light detecting unit 21 of the light detector 20 can provide one dimensional gesture determination such as the hand moving from left to right or from right to left. Using at least three light detecting units 21 of the light detector 20 can provide two dimensional gesture determination toward four directions. Using a light detecting unit matrix of the light detector 20 such as eight by eight matrix can detect image and the position and the moving trace of the finger can be calculated. The movement of the gesture is not limited as aforementioned. The processes can be achieved through different ways and are known in the art so that the details are not described here.

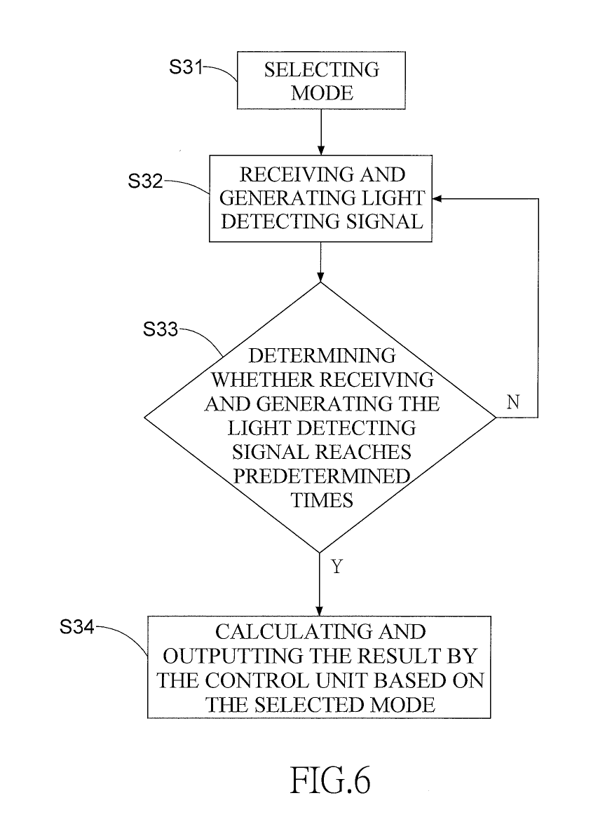

With reference to FIGS. 1, 2 and 6, another performing process comprises following steps. The user selects desired mode through the switching unit 10 (S31). The control unit 40 sends commands to the corresponding light detecting unit 21 to receive light and to generate light detecting signal (S32). The light detecting signal is stored in the data register DR. When the controller 40 reads the light detecting signal stored in the data register DR, the controller 40 determines whether receiving the light detecting signal is reached predetermined time (S33). If the predetermined time is not reached yet, then return to step S32. If the predetermined time is reached, the control unit 40 calculates the parameter based on the received light detecting signals (S34). Then the calculating result is output based on the selected mode.

In specific, the second switching unit 10B is actuated to select an ultraviolet detection mode. the light detecting unit 21 of the light detector 20 receives the ultraviolet in the ambient light to generate an ultraviolet detecting signal. the control unit 40 calculates the intensity of the ultraviolet in the ambient light and outputs the calculating result. The user may determines what to wear or whether go outdoor or not based on the calculated result. The processes to detect the ultraviolet can be achieved through different ways and are known in the art so that the details are not described here.

Further, the portable electronic device as described may provide a color detection mode and may comprise at least three light detecting units 21 to respectively detect red light, green light and blue light. When the color detection mode is selected through the second switching unit 10B or when the color detection mode is automatically actuated while the display 101 is awaked, the light detecting units 21 receives light to generate color detecting signal. The control unit 40 calculates the ratio of the red light, the green light and the blue light in the ambient light based on the generated color detecting signal and then adjusts the illumination color contrast of the backlight module 102 of the display 101. Therefore, the illumination color contrast of the backlight module 102 of the display 101 is adjusted based on the tone of the ambient light to provide best visual effect. The processes to detect the tone of the ambient light and to adjust the illumination color contrast of the backlight module 102 of the display 101 can be achieved through different ways and are known in the art so that the details are not described here.

Moreover, the portable electronic device as describe may provide an ambient light detection mode. When the second switching unit 10B is actuated to select the ambient light detection mode or when the ambient light detection mode is automatically actuated while the display 101 is awaked, the light detector 20 detects the intensity of the ambient light. The light detecting unit 21 receives light to generate an ambient light detecting signal. The control unit 40 calculates the intensity of the ambient light based on the ambient light detecting signal and then adjusts the illumination intensity of the backlight module 102 of the display 10. Therefore, the illumination intensity of the backlight module 102 of the display 101 is adjusted based on the intensity of the ambient light to provide best visual effect. The processes to detect the intensity of the ambient light and to adjust the illumination intensity of the backlight module 102 of the display 101 can be achieved through different ways and are known in the art so that the details are not described here.

The portable electronic device as described as following advantages. Using the same light sources and the same light detecting units to provide different functions allows the portable electronic device as described provides different modes respectively or simultaneously. With the switching units, the user can switch different modes in one single portable electronic device as described to achieve different functions. Therefore, the portable electronic device as described is more convenient and costs less for providing multiple functions.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A portable electronic device comprising:
a first switching unit to switch the portable electronic device to a blood detection mode;
a first light source to provide a first beam, wherein under the blood detection mode, the first beam is emitted to a first object to be reflected as a reflected light, and the first object is a part of an user's body;
a light detector having at least one light detecting unit, wherein under the blood detection mode, the light detector detects the reflected light to generate a blood detecting signal; and
a control unit connecting to the light detector, wherein under the blood detection mode, the control unit calculates the user's blood pressure based on the blood detecting signal.

2. The portable electronic device as claimed in claim 1, wherein the control unit calculates the user's heart rate, the user's arterial stiffness index and the user's vessel obstruction index based on the blood detecting signal.

3. The portable electronic device as claimed in claim 1 further comprising:
a second switching unit to switch the portable electronic device to a blood oxygen saturation detection mode; and
a second light source to provide a second beam having a wave length different with a wave length of the first beam,
wherein under the blood oxygen saturation detection mode, the first beam and the second beams are emitted respectively to the first object to be reflected as reflected lights, and the light detector detects the reflected lights to generate a blood oxygen saturation detecting signal, and the control unit calculates the blood oxygen saturation index based on the blood oxygen saturation signal.

4. The portable electronic device as claimed in claim 1 further comprising a second switching unit to switch the portable electronic device to a breath ethanol concentration analyzing mode, wherein under the breath ethanol concentration analyzing mode, the first beam is emitted to a breath from the user to generate a reflected light, and the light detector detects the reflected light from the breath to generate an ethanol concentration signal, and the control unit calculates the ethanol concentration based on the ethanol concentration signal.

5. The portable electronic device as claimed in claim 1, wherein under a proximity detection mode, the first beam is emitted to a second object to generate a reflected light, and the light detector detects the reflected light from the second object to generate a proximity detecting signal, and the control unit determines whether the second object approaches the portable electronic device based on the proximity detecting signal.

6. The portable electronic device as claimed in claim 1 further comprising a display having a backlight module, wherein the light detector detects an ambient light around a periphery environment of the portable electronic device to generate an ambient light detecting signal, and the control unit adjusts an illumination intensity of the backlight module of the display based on the ambient light detecting signal.

7. The portable electronic device as claimed in claim 1 further comprising a second switching unit to switch the portable electronic device to a gesture detection mode, wherein under the gesture detection mode, the first beam is emitted to a third object to generate a reflected light, the at least one light detecting unit detects the reflected light to generate a gesture detecting signal, and the control unit determines a gesture based on the gesture detecting signal.

8. The portable electronic device as claimed in claim 1 further comprising a second switching unit to switch the portable electronic device to a gesture detection mode, wherein
the light detector comprises multiple light detecting units; and
under the gesture detection mode, the first beam is emitted to a third object to generate a reflected light, the light detecting units detect the reflected light to generate a gesture detecting signal, and the control unit determines a gesture based on the gesture detecting signal.

9. The portable electronic device as claimed in claim 1 further comprising a second switching unit to switch the portable electronic device to a gesture detection mode and at least one second light source to provide a second beam, wherein
the light detector comprises one light detecting unit; and
under the gesture detection mode, the first beam and the second beam are emitted to a third object to respectively generate reflected lights, the light detecting unit detects the reflected lights to generate a gesture detecting signal, and the control unit determines a gesture based on the gesture detecting signal.

10. The portable electronic device as claimed in claim 7, wherein the gesture corresponds to a command of the control unit.

11. The portable electronic device as claimed in claim 1 further comprising a second switching unit to switch the portable electronic device to an ultraviolet detection mode, wherein under the ultraviolet detection mode, the light detector detects an ultraviolet in an ambient light around a periphery environment of the portable electronic device to generate an ultraviolet detecting signal, and the control unit calculates the intensity of the ultraviolet in the ambient light based on the ultraviolet detecting signal.

12. The portable electronic device as claimed in claim 1 further comprising a display having a backlight module and a second switching unit to switch the portable electronic device to a color detection mode, wherein
the light detector has three light detecting units to respectively detect red light, green light and blue light; and
under the color detection mode, the light detecting units respectively detect red light, green light and blue light in an ambient light around a periphery environment of the portable electronic device to generate a color detecting signal, and the control unit adjusts the illumination color contrast of the backlight module of the display based on the color detecting signal.

13. The portable electronic device as claimed in claim 1, wherein the first light source and the light detector are integrated in a semiconductor package.

14. The portable electronic device as claimed in claim 3, wherein the first light source, the second light source and the light detector are integrated in a semiconductor package.

15. The portable electronic device as claimed in claim 1, wherein the first switching unit is an icon for an application program shown on a display.

16. The portable electronic device as claimed in claim 1, wherein the light detector has a driving unit connected to and controlling the first light source.

17. The portable electronic device as claimed in claim 3, wherein the light detector has two driving units respectively connected to and controlling the first and second light sources.

18. A portable electronic device comprising:
a display having a backlight module;
a first switching unit to switch the portable electronic device to a color detection mode;
a light detector having at least three light detecting units to respectively detect red light, green light and blue light, wherein under the color detection mode, the light detecting units respectively detect red light, green light and blue light in an ambient light around a periphery environment of the portable electronic device to generate a color detecting signal; and
a control unit connecting to the light detector, wherein under the color detection mode, the control unit adjusts the illumination color contrast of the backlight module of the display based on the color detecting signal.

19. A portable electronic device comprising:
a first switching unit to switch the portable electronic device to an ultraviolet detection mode;
a light detector having at least one light detecting unit, wherein under the ultraviolet detection mode, the light detector detects an ultraviolet in an ambient light around a periphery environment of the portable electronic device to generate an ultraviolet detecting signal; and
a control unit connecting to the light detector, wherein under the color detection mode, the control unit calculates the intensity of the ultraviolet in the ambient light based on the ultraviolet detecting signal.
